(19) United States
(12) Patent Application Publication
(10) Pub. No.: US 2025/0332220 A1
(43) Pub. Date: Oct. 30, 2025

(54) USE OF CEREBRAL DOPAMINE NEUROTROPHIC FACTOR FOR AMELIORATING PNEUMONIA

(71) Applicant: National Defense Medical Center, Taipei (TW)

(72) Inventors: Gu-Jiun LIN, Taipei (TW); Kuan-Yin Tseng, Taipei (TW); Chin-Mao Hung, Taoyuan (TW); Tai-Lung Cha, Taipei (TW); Mart Saarma, Helsinki (FI); Mikko Tuomas Airavaara, Helsinki (FI)

(21) Appl. No.: 18/647,166

(22) Filed: Apr. 26, 2024

(51) Int. Cl. A61K 38/18; A61P 11/00; A61P 29/00

(52) U.S. Cl. CPC A61K 38/185; A61P 11/00; A61P 29/00

(57) ABSTRACT

The present invention relates to a method for treating and/or preventing pneumonia, including administering a therapeutically effective amount of cerebral dopamine neurotrophic factor (CDNF) to a subject in need thereof. The pneumonia is caused by infection of influenza A virus or severe acute respiratory syndrome coronavirus-2 (SARS-CoV-2).

### SUBMISSION OF SEQUENCE LISTING AS ASCII TEXT FILE

This application includes an electronically submitted sequence listing in XML format. The XML file contains a sequence listing entitled “P24-0007US_Sequence_Listing.xml” which was created on Apr. 26, 2024 and is 1,877 bytes in size. The sequence listing contained in this XML file is part of the specification and is hereby incorporated by reference herein in its entirety.

### BACKGROUND OF THE INVENTION

### 1. Field of the Invention

The present invention relates to the use of cerebral dopamine neurotrophic factor (CDNF) for the treatment and/or prevention of pneumonia, especially to the utilization of cerebral dopamine neurotrophic factor via immunomodulatory mechanisms to ameliorate pneumonia and related symptoms caused by respiratory virus infections.

### 2. Description of the Prior Art

Pneumonia is a respiratory infection disease characterized by inflammation in the lungs. Common symptoms of pneumonia include cough, fever, rapid breathing, and chest pain. It is typically caused by viral, bacterial, fungal, or microbial infections.

In recent years, with the outbreak of COVID-19 and influenza A virus, pneumonia caused by respiratory virus infections has attracted wide attention. Viruses such as influenza A virus or novel coronaviruses (SARS-CoV-2) use host cells to produce a large number of viral proteins and induce a cytokine storm, resulting in an overreaction of the immune system that cannot be properly regulated. These factors cause severe pneumonia in the host and the death of the host.

Currently, there is no effective treatment for pneumonia caused by influenza or novel coronaviruses in clinical practice. Existing treatments often fail to achieve significant therapeutic effects, especially in cases of severe pneumonia. As a result, pneumonia induced by respiratory viruses frequently leads to a high patient mortality rate. Therefore, there is an urgent need for an effective treatment of symptoms associated with severe pneumonia.

### SUMMARY OF THE INVENTION

In one aspect, the present invention provides a method for treating and/or preventing pneumonia, comprising administering a therapeutically effective amount of cerebral dopamine neurotrophic factor (CDNF) to a subject in need thereof.

In another aspect, the present invention provides a method for reduction of an inflammatory response caused by infection of a virus, comprising administering a therapeutically effective amount of cerebral dopamine neurotrophic factor to a subject in need thereof.

In another aspect, the present invention provides a method for treating and/or preventing pneumonia, comprising administering a composition comprising a therapeutically effective amount of cerebral dopamine neurotrophic factor to a subject in need thereof.

These and other aspects will become apparent from the following description of the preferred embodiment taken in conjunction with the following drawings.

### BRIEF DESCRIPTION OF THE DRAWINGS

The accompanying drawings illustrate one or more embodiments of the invention and, together with the written description, serve to explain the principles of the invention. Wherever possible, the same reference numbers are used throughout the drawings to refer to the same or like elements of an embodiment.

FIG. 1 shows mouse survival rates within 21 days post-infection with influenza virus. Mice were challenged with 500 pfu (2×LD₅₀) of influenza A virus PR8 strain and then treated with 10 μg/mouse of rhCDNF (SEQ ID NO: 1) from 1 to 5 days post-infection (CDNF group, n=10) or treated with phosphate buffered saline (PBS) as a vehicle control group (PBS group, n=8). The survival rate for each group was recorded daily for 21 days post-infection. *p<0.05.

FIGS. 2A to 2C show histopathological analysis of leukocyte infiltration in lungs of mice at 6 days post-infection with influenza virus. Mice without virus infection (FIG. 2A, Uninf group) were used as negative control. In addition, mice were challenged with 500 pfu (2×LD₅₀) of influenza A virus PR8 strain and then treated with PBS (FIG. 2B, PBS group) or treated with 10 μg/mouse of rhCDNF (SEQ ID NO: 1) (FIG. 2C, CDNF group) from 1 to 5 days post-infection. The scale bar is 50 μm.

FIGS. 3A to 3C show the levels of pro-inflammatory cytokines, IL-10 (FIG. 3A) and TNF-α (FIG. 3B), and anti-inflammatory cytokine, IL-10 (FIG. 3C), in lungs of mice at 6 days post-infection with influenza virus. Bronchial alveolar lavage fluid (BALF) from mice without virus infection (Uninf group), mice treated with PBS for 5 days after challenged with 500 pfu (2×LD₅₀) of influenza A virus PR8 strain (PBS group), and mice treated with 10 μg/mouse of rhCDNF (SEQ ID NO: 1) for 5 days after challenged with 500 pfu (2×LD₅₀) of influenza A virus PR8 strain (CDNF group) were quantified for the amounts of cytokines in an enzyme linked immunosorbent assay (ELISA). *p<0.05, **p<0.01, ***p<0.001.

FIGS. 4A to 4C show the population of IL-10-producing CD4⁺ T cells (IL-10⁺CD4⁺; FIG. 4A), IL-17-producing CD4⁺ T cells (IL-17⁺CD4⁺; FIG. 4B), and regulatory T cells (T_{reg}, Foxp3⁺CD25⁺; FIG. 4C) in spleen of mice at 6 days post-infection with influenza virus. Lymphocyte samples collected from mice in FIGS. 2A and 2B were subjected to flow cytometry analysis. *p<0.05, **p<0.01.

FIG. 5 shows body weight change of hamsters within 6 days post-infection with SARS-CoV-2. Hamsters were challenged with 0.05 ml of 1×10⁵ PFU/ml wild type SARS-CoV-2 and then treated with 40 μg/hamster of rhCDNF (SEQ ID NO: 1) from 1 to 5 days post-infection (SARS-CoV-2+CDNF group, n=6) or treated with PBS as a vehicle control group (SARS-CoV-2+PBS group, n=6). Line plots show mean±standard error of the mean (SEM). Statistical significance was calculated with Student's t-test. *p<0.05, **p<0.01.

FIGS. 6A to 6C show histopathological analysis of leukocyte infiltration in lungs of hamster at 7 days post-infection with SARS-CoV-2. Hamsters treated with 40 μg/hamster of rhCDNF (SEQ ID NO: 1) without SARS-CoV-2 challenge were used as negative control (FIG. 6A, CDNF group). Hamsters treated with PBS (FIG. 6B, SARS-CoV-2+PBS group) or treated with 40 μg/hamster of rhCDNF (SEQ ID NO: 1) (FIG. 6C, SARS-CoV-2+CDNF group) from 1 to 5 days post-infection were also subjected to histopathological analysis. The scale bar is 50 μm.

### DETAILED DESCRIPTION OF THE PREFERRED EMBODIMENT

The present invention relates to using CDNF for treating pneumonia, demonstrating significant efficacy in reducing pulmonary inflammation and effectively alleviating the symptoms of pneumonia. Specifically, for severe pneumonia caused by respiratory viruses such as SARS-CoV-2 and influenza viruses, the present invention shows that CDNF can effectively regulate the inflammatory response, thereby reducing the mortality rates caused by viral infection.

Therefore, the present invention relates to a method for treating and/or preventing pneumonia, comprising administering a therapeutically effective amount of CDNF to a subject in need thereof.

The present invention also relates to a method for reduction of an inflammatory response caused by infection of a virus, comprising administering a therapeutically effective amount of cerebral dopamine neurotrophic factor (CDNF) to a subject in need thereof.

The present invention further relates to a method for treating and/or preventing pneumonia, comprising administering a composition comprising a therapeutically effective amount of cerebral dopamine neurotrophic factor to a subject in need thereof.

In some embodiments, the CDNF comprises a functional fragment of CDNF. In some embodiments, the CDNF comprises a polypeptide sequence at least 90%, 91%, 92%, 93%, 94%, 95%, 96%, 97%, 98%, 99%, or 100% identical to SEQ ID NO: 1.

In some embodiments, the pneumonia is caused by infection of a virus. In some embodiments, the virus is one of influenza A virus and severe acute respiratory syndrome coronavirus 2 (SARS-CoV-2).

In some embodiments, the inflammatory response is caused by infection of influenza A virus or severe acute respiratory syndrome coronavirus 2 (SARS-CoV-2).

In some embodiments, the reduction of the inflammatory response comprises decrease of pro-inflammatory cytokine, increase of anti-inflammatory cytokine, and/or reduction of leukocyte infiltration. In some embodiments, the pro-inflammatory cytokine comprises at least one of interleukin-1β (IL-1β), tumor necrosis factor-α (TNF-α), and interleukin-17 (IL-17). In some embodiments, the anti-inflammatory cytokine comprises interleukin-10 (IL-10).

In some embodiments, the reduction of the inflammatory response comprises at least one of a decrease of a population of IL-17-producing T cells, an increase of a population of IL-10-producing T cells, and an increase of a population of regulatory T cells.

In some embodiments, the composition is able to decrease pro-inflammatory cytokines, increase anti-inflammatory cytokines, and/or reduce leukocyte infiltration. In some embodiments, the pro-inflammatory cytokine comprises at least one of interleukin-1p (IL-1p), tumor necrosis factor-α (TNF-α), and interleukin-17 (IL-17). In some embodiments, the anti-inflammatory cytokine comprises interleukin-10 (IL-10).

In some embodiments, the composition is able to decrease a population of IL-17-producing T cells, increase a population of IL-10-producing T cells, and increase a population of regulatory T cells.

In some embodiments, the composition further comprises at least one pharmaceutically acceptable carrier, adjuvant, excipient, and/or diluent.

In some embodiments, the composition is administered via a route selected from the group consisting of oral, topical, transdermal, parenteral, subcutaneous, intranasal, intratracheal, intrabronchial, mucosal, intramuscular, intraperitoneal, intravitreal, and intravenous routes.

In some embodiments, the composition is in the form of a tablet, dragee, liquid, drop, suppository, capsule, caplet, or gelcap.

It should be understood that both the foregoing general description and the following detailed description are exemplary and explanatory but are not restrictive of the invention as claimed. Certain details of one or more embodiments of the invention are set forth in the description below. Other features or advantages of the present invention will be apparent from the non-exhaustive list of representative examples that follows, and also from the appending claims.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The methods and techniques of the present disclosure are generally performed according to conventional methods well-known in the art. Generally, nomenclatures used in connection with, and techniques of biochemistry, enzymology, molecular and cellular biology, microbiology, genetics, and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification unless otherwise indicated.

As used herein, the singular form “a”, “an”, and “the” includes plural references unless indicated otherwise. For example, “an” excipient includes one or more excipients.

As used interchangeably herein, “around”, “about” and “approximately” shall generally mean plus or minus 10% of the numerical value of the number with which it is being used. Therefore, about 1% means in the range of 0.9% to 1.1%. Numerical quantities given herein are approximate, meaning that the term “around”, “about” or “approximately” can be inferred if not expressly stated.

As used herein, the phrase “comprising” is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments.

Where applicants have defined an invention or a portion thereof with an open-ended term such as “comprising,” it should be readily understood that (unless otherwise stated) the description should be interpreted to also describe such an invention using the terms “consisting essentially of” or “consisting of.”

As used herein, the term “treat,” “treating,” or “treatment” encompasses alleviation of at least one symptom thereof, a reduction in the severity thereof, or inhibition of the progression thereof. Treatment does not necessarily mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the term “prevent,” “preventing,” or “prevention” refers to being able to substantially preclude, avert, obviate, forestall, stop, hinder, or a combination thereof, any aspect of a disease, condition, or combination thereof from happening, especially by advance action.

As used herein, the term “subject” refers to an animal, more particularly to non-human mammals and human organisms. Non-human animal subjects may also include prenatal forms of animals, such as, e.g., embryos or fetuses. Non-limiting examples of non-human animals include horse, cow, camel, goat, sheep, dog, cat, non-human primate, mouse, rat, rabbit, hamster, guinea pig, and pig. In some embodiments, the subject is a human. Human subjects may also include fetuses. As used herein, the term “subject,” refers to any subject, particularly a mammalian subject, for whom therapy is desired, for example, a human.

As used herein, the term “an effective amount,” “a sufficient amount,” or “a therapeutically effective amount,” which can be used interchangeably, of a substance is that amount sufficient to effect beneficial or desired results, including clinical results. Specifically, it refers to a dosage sufficient to alleviate symptoms of pneumonia, such as coughing, fever, rapid breathing, viral shedding, or detectable pneumonia.

As used herein, the term “Cerebral dopamine neurotrophic factor (CDNF)” refers to a neurotrophic factor protein. CDNF is a small monomeric protein with a molecular weight of approximately 18 kDa that is expressed in the central nervous system but also in nonneuronal tissues. The full-length human CDNF, which has a total length of 187 amino acids comprises an N-terminal signal peptide that directs them to the ER. The mature human CDNF without the N-terminal signal peptide has a total length of 161 amino acids. Notably, CDNF contains a C-terminal KDEL-like ER-retention signal that is typically absent in growth factors destined for secretion. CDNF accumulates in the ER lumen in healthy cells and disruption of the C-terminal ER-retention signal results in their secretion. Detectable levels of CDNF can be found in normal human serum. CDNF is a conserved protein in vertebrates and invertebrates, exhibiting neuroprotective functions. Previous research has shown that CDNF plays a significant role in neurodegenerative diseases within the nervous system, such as Parkinson's disease.

As used herein, the term “functional fragment” refers to a fragment that retains the biological activity of a complete polypeptide. Specifically, the functional fragment used herein refers to a peptide fragment of CDNF that has the activity of treating and/or preventing pneumonia. In some embodiments, the functional fragment of CDNF described in the present invention removes its N-terminal signaling peptide. CDNF described in the present invention refers to U.S. Patent Publication No. 2019/0192629, the entirety of which is herein incorporated by reference. Unless otherwise defined, the CDNF used in the examples of the present invention is a recombinant human cerebral dopamine neurotrophic factor (rhCDNF). In some embodiments, the amino acid sequence of the CDNF described in the present invention has at least 90%, 91%, 92%, 93%, 94%, 95%, 96%, 97%, 98%, 99%, or 100% sequence identity to SEQ ID NO: 1.

As used herein, the term “pro-inflammatory cytokines” refers to protein molecules that promote inflammatory responses, primarily released by immunocytes such as macrophages, T cells, and B cells. Examples of pro-inflammatory cytokines include, but are not limited to, tumor necrosis factor (TNF-α), interleukin-1β (IL-1β), interleukin-6 (IL-6), interleukin-8 (IL-8), interleukin-17 (IL-17), and interferon (IFN-γ). As used herein, the term “anti-inflammatory cytokines” refers to immunoregulatory molecules that limit the sustained or excessive inflammatory response and control the pro-inflammatory cytokine response. Examples of anti-inflammatory cytokines include, but are not limited to, transforming growth factor β (TGF-β), interleukin-4 (IL-4), interleukin-10 (IL-10), interleukin-11 (IL-11), and interleukin-13 (IL-13).

As used herein, the terms “decrease,” “increase,” “reduce,” or “promote” refer to changes in expression level or physiological phenomena compared to subjects who are not administered the CDNF described in the present invention.

As used herein, the phrase “pharmaceutically acceptable” refers to those compounds, materials, compositions, and/or dosage forms which are, within the scope of sound medical judgment, suitable for use in contact with the tissues of human beings, animals, and plants without excessive toxicity, irritation, allergic response, or other problem or complication, commensurate with a reasonable benefit/risk ratio.

As used herein, “pharmaceutically acceptable carrier,” “pharmaceutically acceptable adjuvant,” “pharmaceutically acceptable excipient,” or “pharmaceutically acceptable diluent” includes any and all solvents, dispersion media, coatings, antibacterial and antifungal agents, isotonic and absorption enhancing or delaying agents, and other excipients or additives that are physiologically compatible. In specific embodiments, the carrier is suitable for intranasal, intravenous, intramuscular, intradermal, subcutaneous, parenteral, oral, transmucosal, or transdermal administration. Depending on the route of administration, the active compound may be coated in a material to protect the compound from the action of acids and other natural conditions which may inactivate the compound. The use of such media and agents for pharmaceutically active substances is well-known in the art.

The composition of the present invention may be administered to subjects by a variety of administration routes, including intradermal, intramuscular, subcutaneous, intravenous, intra-atrial, intra-articular, intraperitoneal, parenteral, oral, rectal, intranasal, intrapulmonary, and transdermal delivery, or topically to the eyes, ears, skin or mucous membranes. Alternatively, CDNF may be administered ex-vivo by direct exposure to cells, tissues, or organs originating from a subject (autologous) or another subject (allogeneic), optionally in a biologically suitable, liquid or solid carrier.

Formulations suitable for administration of the present invention may comprise, possibly among other things well known to those of skill in the art: aqueous and non-aqueous solutions, antioxidants, bacteriostats, buffers, solutes that affect isotonicity, preservatives, solubilizers, stabilizers, suspending agents, thickening agents, or a combination thereof. In some embodiments, the composition is in the form of a tablet, dragee, liquid, drop, suppository, capsule, caplet, or gelcap.

Additionally, or as an alternative approach, formulations suitable for administration of the present invention may comprise, possibly among other things well known to those of skill in the art: gels, PEG such as PEG 400, propylene glycol, saline, sachets, water, other appropriate liquids known in the art, or a combination thereof.

Also in the addition or in the alternative, formulations suitable for administration of the present invention may comprise, possibly among other things well known to those of skill in the art: binders, buffering agents, calcium phosphates, cellulose, colloids, such as colloidal silicon dioxide, colorants, diluents, disintegrating agents, dyes, fillers, flavoring agents, gelatin, lactose, magnesium stearate, mannitol, microcrystalline gelatin, moistening agents, paraffin hydrocarbons, pastilles, polyethylene glycols, preservatives, sorbitol, starch, such as corn starch, potato starch, or a combination thereof, stearic acid, sucrose, talc, triglycerides, or a combination thereof.

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### EXAMPLES

### Example 1 Effect of CDNF on Pneumonia Caused by Influenza Virus Infection

In this example, CDNF described in the present invention was administered to a mouse model of influenza virus infection to investigate the effect of CDNF on pneumonia caused by influenza virus infection.

### Materials and Methods

Animal tests in all of the Examples in the present invention were approved by the Institutional Animal Care and Use Committee (IACUC) of National Defense Medical Center (Taipei, Taiwan) in accordance with the Guide for the Care and Use of Laboratory Animals issued by the National Institutes of Health (NIH) (MD, USA).

Virus production and titration. Influenza H1N1 A/PR/8/34 (PR8) virus was produced by transfecting 293T cells with plasmids expressing the components of PR8 virus. The obtained virus was then amplified in Madin-Darby canine kidney (MDCK) cells. For virus titration, tenfold serial dilution of the prepared influenza virus was added to 90% confluent MDCK cells, and the mixture was incubated for 1 hour. After incubation, the cells were washed with phosphate-buffered saline (PBS) and then covered with 0.6% agarose gel containing 1× serum-free DMEM.

Mouse model of influenza virus infection and CNDF treatment. C57BL/6 mice (National Laboratory Animal Center, Taipei, Taiwan) were anesthetized by isoflurane and challenged intranasally with 25 μl of 20 pfu/μl influenza A virus PR8 strain (H1N1) suspension. The 50% lethal dose (LD₅₀) of the influenza A virus in mice was around 250 pfu, and therefore, the mice were challenged with 500 pfu (2×LD₅₀) influenza virus. For CDNF treatment, mice infected with PR8 virus were subcutaneously injected with 10 μg/mouse of rhCDNF (SEQ ID NO: 1) from 1 to 5 days post-infection (d.p.i.) (CDNF group). Virus-infected mice injected with PBS were used as a vehicle control group (PBS group). The survival rate for each group was recorded daily after infection for 21 days.

Histopathological analysis (leukocyte infiltration). Mice were sacrificed at 6 days post-infection, and lungs were harvested and then embedded in parafilm. Sections of 6 μm thickness were cut and stained with hematoxylin and eosin (H&E) staining and then analyzed under a light microscopy to observe leukocyte infiltration.

Enzyme-linked immunosorbent assay (EISA) (cytokines analysis). Mice were sacrificed at 6 days post-infection, and bronchial alveolar lavage fluid (BALF) was collected for cytokine analysis (IL-1β, TNF-α, and IL-10) using ELISA commercial assay kits (Biolegend, San Diego, CA, USA; IL-1β, catalog no. 432604; IL-10, catalog no. 431414; TNF-α, catalog no. 430904). Briefly, capture antibody against IL-10, TNF-α, or IL-10 was initially coated on an ELISA plate and incubated at 4° C. overnight. After removing uncoated antibodies, samples were added to the plate to incubate with the capture antibody for 2 hours at room temperature (RT). After removing unbound samples, detection antibody against IL-1β, TNF-α, or IL-10 was added to the plate to incubated with the reaction for 1 hour, RT. After removing unbound detection antibody, avidin-horseradish peroxidase (HRP) was added to the plate to incubate with the reaction for 30 minutes, RT. After removing excess avidin-HRP, substrate was added to the plate for color development. After the development was stopped, cytokine concentration in the samples was quantified by measuring absorbance (optical density, O.D.) at 450 nm and/or 570 nm.

Flow cytometry analysis. Mice were sacrificed at 6 days post-infection, and lymphocyte samples were collected from spleen for flow cytometry analysis to detect levels of T cells producing different cytokines. Lymphocyte samples were stained with allophycocyanin (APC)-conjugated anti-mouse CD4 antibody (clone GK1.5, eBioscience, San Diego, CA, USA), and/or phycoerythrin (PE)-conjugated anti-mouse CD25 antibody (clone PC61, eBioscience) for 30 min at 4° C. For Foxp3 staining, cells were stained with surface molecules, fixed and permeabilized overnight with Fixation/Permeabilization working solution. After fixation and permeabilization, cells were stained with FITC-conjugated anti-Foxp3 (clone FJK-16S, eBioscience). For intracellular cytokine staining, cells were stimulated for 4-6 hours with 20 ng/mL phorbol 12-myristate 13-acetate and 1 μM ionomycin in the presence of 4 μM monensin. Intracellular Fixation and Permeabilization kit (eBioscience, catalog no. 88-8824-00) was used for intracellular cytokine staining. Stimulated cells were stained with APC-conjugated anti-mouse CD4 antibody (BioLegend, clone GK1.5; BD Biosciences, clone RM4-5, Franklin Lakes, NJ, USA) on ice for 25-30 minutes in the dark, washed with 1 ml of FACS buffer (PBS containing 0.5% FBS), and fixed with 0.1 ml of IC Fixation Buffer for 20 minutes at room temperature in the dark. After washing with 1 ml of Permeabilization Buffer, cells were resuspended in 80 μl of Permeabilization Buffer and incubated at room temperature for 10 minutes. Cells were stained with FITC-conjugated anti-IL-10 antibody (eBioscience, clone JES5-16E3) for 20 min, RT. Flow cytometric analysis was performed with FACSCalibur™ Flow Cytometer (BD Biosciences) and CellQuest software (BD Biosciences).

Statistical analysis. Experimental results were analyzed using Student's t-test to evaluate the significant difference between groups (*p<0.05, **p<0.01, ***p<0.001).

### Results

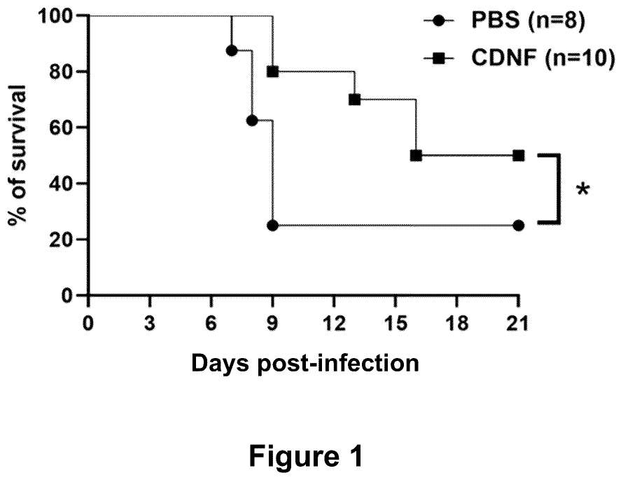

CDNF reduces mortality caused by influenza virus infection. As shown in FIG. 1, the survival rate of the treatment group (CDNF group, n=10) at 21 days post-infection was approximately 50%, whereas the survival rate of the control group (PBS group, n=8) was only 25%. The CDNF group exhibited a significantly higher survival rate compared to the PBS group, indicating that CDNF reduces mortality caused by influenza virus infection.

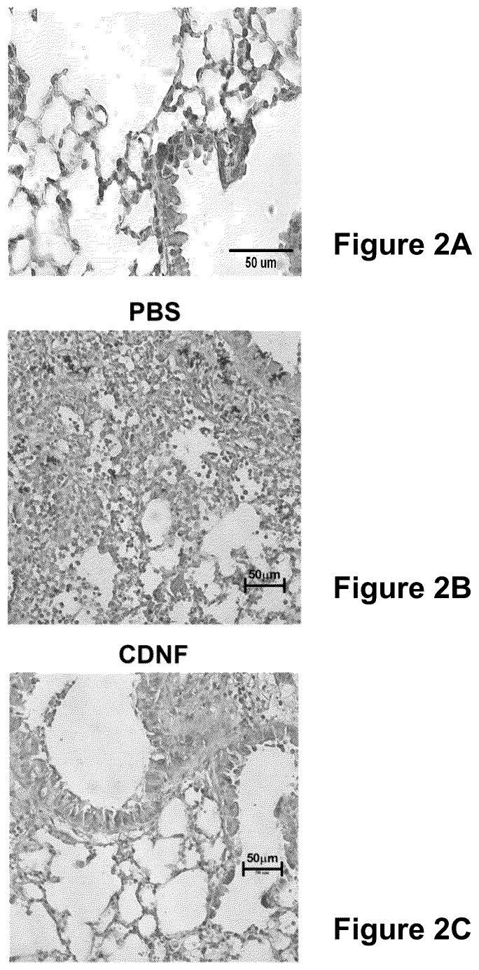

CDNF reduces leukocyte infiltration caused by influenza virus infection. As shown in FIG. 2A, no leukocyte infiltration was observed in the lung tissues of mice without virus infection (Uninf group). As shown in FIG. 2B, leukocyte infiltration was observed in the lung tissues of mice treated with PBS after infected with influenza virus (PBS group). In contrast, as shown in FIG. 2C, there was an significant reduction in leukocyte infiltration in the lung tissues of mice treated with CDNF after infected with influenza virus (CDNF group). Leukocyte infiltration is a physiological phenomenon in which white blood cells penetrate the blood vessel walls and enter tissues or organs. This phenomenon is commonly observed during inflammation, indicating inflammation-related responses. The results indicate that CDNF effectively reduces leukocyte infiltration and thus inhibits inflammatory response and treats and/or prevents pneumonia.

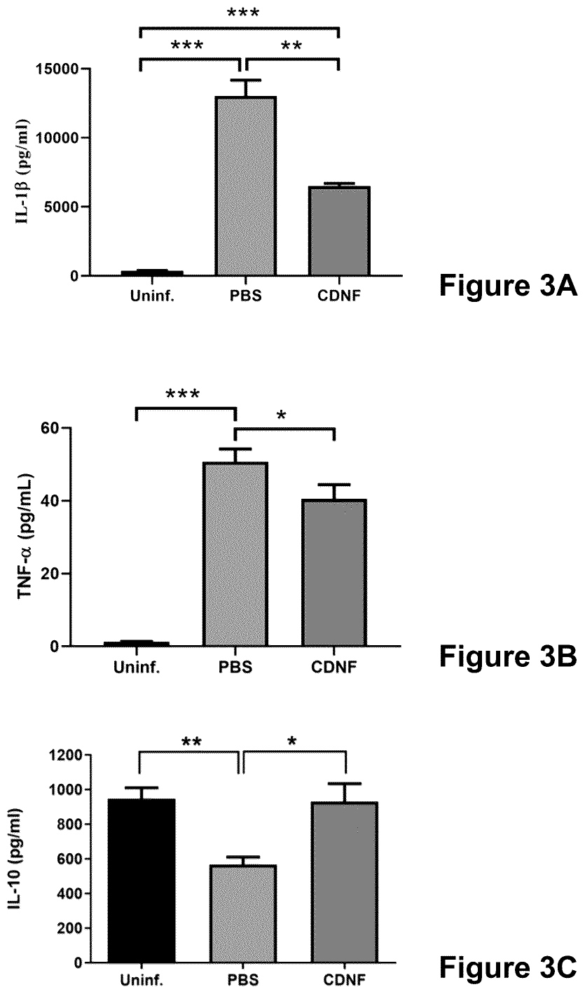

CDNF inhibits pneumonia caused by influenza virus infection through reducing pro-inflammatory cytokines in lungs. As shown in FIGS. 3A and 3B, the levels of IL-10 (FIG. 3A) and TNF-α (FIG. 3B), two pro-inflammatory cytokines, in lungs of mice treated with PBS after infected with influenza virus (PBS group) were significantly elevated (p<0.001) compared to those of mice without virus infection (Uninf group), indicating that influenza virus infection causes a significant increase in pro-inflammatory cytokines in lungs, which facilitates the progression of inflammation and leads to pneumonia. In contrast, the levels of IL-1β and TNF-α in lungs of mice treated with CDNF after infected with influenza virus (CDNF group) were significantly decreased (p<0.01 or p<0.05) compared to those of the PBS group, demonstrating that CDNF effectively reduces the expression of pro-inflammatory cytokines in lungs after influenza virus infection, thereby inhibiting inflammatory response and alleviating/treating pneumonia symptoms caused by influenza virus.

CDNF inhibits pneumonia caused by influenza virus infection through restoring anti-inflammatory cytokines in lungs. As shown in FIG. 3C, the level of IL-10, an anti-inflammatory cytokine, in lungs of the PBS group mice was significantly decreased (p<0.01) compared to that of the Uninf group mice, indicating that influenza virus infection causes a significant reduction in anti-inflammatory cytokines in lungs, which facilitates the progression of inflammation and leads to pneumonia. In contrast, the level of IL-10 in lungs of CDNF group mice was significantly increased (p<0.05) compared to that of the PBS group mice and restored to the same level as that of the Uninf group mice, indicating that CDNF effectively promotes the expression of anti-inflammatory cytokines in lungs after influenza virus infection, thereby inhibiting inflammatory response and alleviating/treating pneumonia symptoms caused by influenza virus.

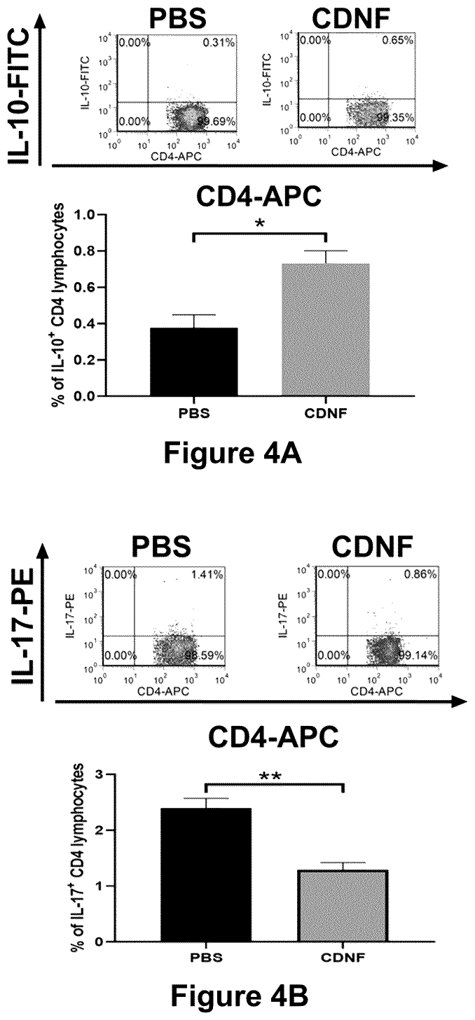

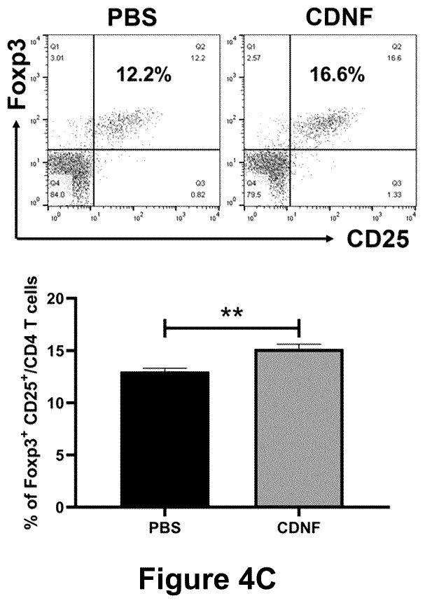

CDNF inhibits pneumonia caused by influenza virus infection through increasing anti-inflammatory cytokine-producing T cells and decreasing pro-inflammatory cytokine-producing T cells. As shown in FIG. 4A, the proportion of IL-10-producing CD4⁺ T cells (IL-10⁺CD4⁺) among total CD4⁺ T cells in the PBS group (control) was approximately 0.31%, whereas the proportion of IL-10-producing CD4⁺ T cells among total CD4⁺ T cells in the CDNF group was approximately 0.65%. The result indicates a significant increase (p<0.05) in the level of IL-10-producing CD4⁺ T cells in the CDNF group compared to the PBS control group. In addition, as shown in FIG. 4B, the proportion of IL-17-producing CD4⁺ T cells among total CD4⁺ T cells in the PBS control group was approximately 1.41%, whereas the proportion of IL-17-producing CD4⁺ T cells among total CD4⁺ T cells in the CDNF group was approximately 0.86%. The result indicates a significant decrease (p<0.01) in the level of IL-17-producing CD4⁺ T cells in the CDNF group compared to the PBS control group. These results demonstrate that administering CDNF to mice with pneumonia caused by influenza virus infection effectively decreases the level of IL-17-producing CD4⁺ T cells and increases the level of IL-10-producing CD4⁺ T cells, leading to a decrease of IL-17, a pro-inflammatory cytokine, and an increase of IL-10, an anti-inflammatory cytokine, thereby inhibiting inflammatory response and alleviating/treating pneumonia symptoms caused by influenza virus.

CDNF inhibits pneumonia caused by influenza virus infection through promoting the generation of regulatory T cells. As shown in FIG. 4C, the proportion of regulatory T cells (T_{reg}, Foxp3⁺CD25⁺) among total CD4⁺ lymphocytes in the PBS group (control) was approximately 12.2%, whereas the proportion of regulatory T cells among total lymphocytes in the CDNF group was approximately 16.6%. The result indicates a significant increase (p<0.01) in the level of regulatory T cells in the CDNF group compared to the PBS control group. Regulatory T cells inhibit the activity of other immune cells to maintain the balance of the immune system and prevent excessive immune responses. The results demonstrate that administering CDNF to mice with pneumonia caused by influenza virus infection effectively promotes the generation of regulatory T cells, thereby inhibiting inflammatory response and alleviating pneumonia symptoms caused by influenza virus..

### Example 2 Effect of CDNF on Pneumonia Caused by SARS-CoV-2 Infection

In this example, CDNF described in the present invention was administered to a hamster model of SARS-CoV-2 infection to investigate the effect of CDNF on pneumonia caused by SARS-CoV-2 infection.

### Materials and Methods

Hamster model of SARS-CoV-2 infection and CNDF treatment. Golden Syrian hamsters (National Laboratory Animal Center, Taipei, Taiwan) were challenged with 0.05 ml of 1×10⁵ PFU/ml wild type SARS-CoV-2 (hCoV-19/Taiwan/4/2020, GISAID accession number: EPI_ISL_411927) by intratracheal injection. For CDNF treatment, hamsters infected with SARS-CoV-2 were subcutaneously injected with 40 μg/hamster of rhCDNF (SEQ ID NO: 1) from 1 to 5 days post-infection (SARS-CoV-2+CDNF group). SARS-CoV-2 infected hamsters injected with PBS were used as vehicle control (SARS-CoV-2+PBS group). In addition, hamsters subcutaneously injected with 40 μg/hamster of rhCDNF (SEQ ID NO: 1) without SARS-CoV-2 challenge were used as negative control (CDNF group). The body weight of hamsters in each group was measured daily after infection for 6 days.

Histopathological analysis (leukocyte infiltration). Hamster were sacrificed at 7 days post-infection, and lungs were harvested and then embedded in parafilm. Sections of 6 μm thickness were cut and stained with H&E staining and then analyzed under a light microscopy to observe leukocyte infiltration.

Statistical analysis. Methods of statistics are the same as described in Example 1.

### Results

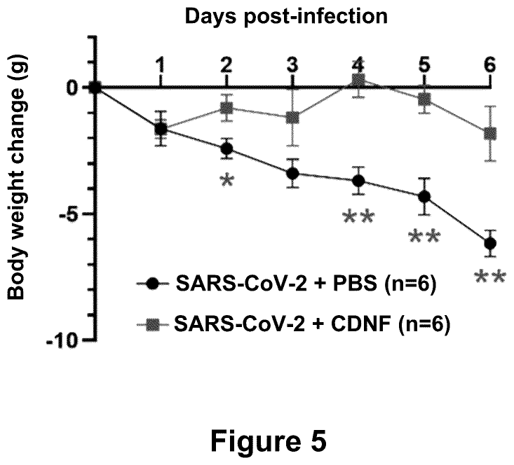

CDNF has a protective effect on COVID-19 through alleviating weight loss caused by SARS-CoV-2 infection. As shown in FIG. 5, hamsters treated with PBS after SARS-CoV-2 infection (SARS-CoV-2+PBS group) experienced progressively severe weight loss at 6 days post-infection. In contrast, hamsters treated with CDNF after SARS-CoV-2 infection (SARS-CoV-2+CDNF group) did not experienced significant weight loss. In particular, the body weight of the SARS-CoV-2+CDNF group was significantly higher than that of the SARS-CoV-2+PBS group at 2, 4, 5, and 6 days post-infection (p<0.05 or p<0.01). The result indicates that CDNF has a protective effect on COVID-19 through alleviating weight loss caused by SARS-CoV-2 infection.

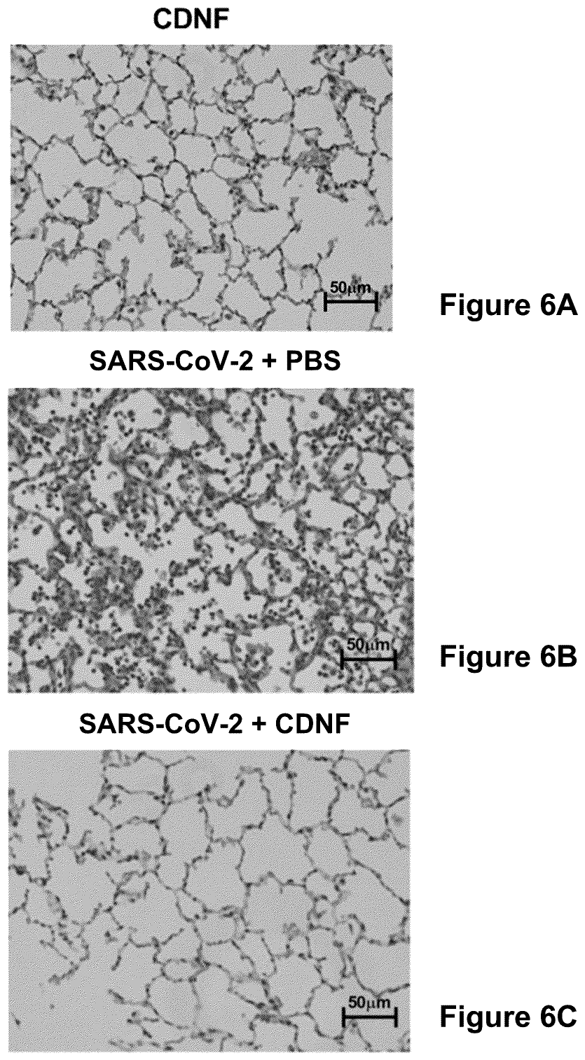

CDNF has no adverse effects on subjects receiving it and reduces leukocyte infiltration caused by SARS-CoV-2 infection. As shown in FIG. 6A, no leukocyte infiltration was observed in the lung tissues of hamsters treated with CDNF without SARS-CoV-2 infection (CDNF group). As shown in FIG. 6B, leukocyte infiltration was observed in the lung tissues of hamsters treated with PBS after infected with SARS-CoV-2 (SARS-CoV-2+PBS group). In contrast, as shown in FIG. 6C, there was an significant reduction in leukocyte infiltration in the lung tissues of hamsters treated with CDNF after infected with SARS-CoV-2 (SARS-CoV-2+CDNF group). The results indicate that CDNF has no adverse effects on subjects receiving it. The results also demonstrate that CDNF effectively reduces leukocyte infiltration caused by SARS-CoV-2 infection and thus inhibits inflammatory response and treats and/or prevents pneumonia.

In summary, the present invention demonstrates that CDNF inhibits pneumonia caused by virus infection through decreasing pro-inflammatory cytokine-producing T cells, increasing anti-inflammatory cytokine-producing T cells and regulatory T cells, reducing pro-inflammatory cytokines (IL-1β and TNF-α) in lungs, restoring anti-inflammatory cytokine (IL-10) in lungs, and reducing leukocyte infiltration in lungs, thereby decreasing mortality caused by virus infection. Additionally, CDNF has a protective effect on COVID-19 through alleviating weight loss caused by SARS-CoV-2 infection and reducing leukocyte infiltration in lungs. The present invention also demonstrates that CDNF possesses no adverse effects on subjects receiving it. These data indicate a great potential of CDNF for treating and/or preventing pneumonia caused by viral infection in clinical application.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

What is claimed is:

1. A method for treating and/or preventing pneumonia, comprising administering a therapeutically effective amount of cerebral dopamine neurotrophic factor (CDNF) to a subject in need thereof.

2. The method of claim 1, wherein the cerebral dopamine neurotrophic factor comprises a polypeptide sequence at least 90%, 91%, 92%, 93%, 94%, 95%, 96%, 97%, 98%, 99%, or 100% identical to SEQ ID NO: 1.

3. The method of claim 1, wherein the pneumonia is caused by infection of a virus.

4. The method of claim 3, wherein the virus is one of influenza A virus and severe acute respiratory syndrome coronavirus 2 (SARS-CoV-2).

5. A method for reduction of an inflammatory response caused by infection of a virus, comprising administering a therapeutically effective amount of cerebral dopamine neurotrophic factor (CDNF) to a subject in need thereof.

6. The method of claim 5, wherein the cerebral dopamine neurotrophic factor comprises a polypeptide sequences at least 90%, 91%, 92%, 93%, 94%, 95%, 96%, 97%, 98%, 99%, or 100% identical to SEQ ID NO: 1.

7. The method of claim 5, wherein the virus is one of influenza A virus and severe acute respiratory syndrome coronavirus 2 (SARS-CoV-2).

8. The method of claim 5, wherein the reduction of the inflammatory response comprises decrease of pro-inflammatory cytokine, increase of anti-inflammatory cytokine, and/or reduction of leukocyte infiltration.

9. The method of claim 8, wherein the pro-inflammatory cytokine comprises at least one of interleukin-1β (IL-1β) and tumor necrosis factor-α (TNF-α).

10. The method of claim 8, wherein the anti-inflammatory cytokine comprises interleukin-10 (IL-10).

11. The method of claim 5, wherein the reduction of the inflammatory response comprises at least one of a decrease of a population of IL-17-producing T cells, an increase of a population of IL-10-producing T cells, and an increase of a population of regulatory T cells.

12. A method for treating and/or preventing pneumonia, comprising administering a composition comprising a therapeutically effective amount of cerebral dopamine neurotrophic factor to a subject in need thereof.

13. The method of claim 12, wherein the cerebral dopamine neurotrophic factor comprises a polypeptide sequence at least 90%, 91%, 92%, 93%, 94%, 95%, 96%, 97%, 98%, 99%, or 100% identical to SEQ ID NO: 1.

14. The method of claim 12, wherein the pneumonia is caused by infection of a virus.

15. The method of claim 12, wherein the virus is one of influenza A virus and severe acute respiratory syndrome coronavirus 2 (SARS-CoV-2).

16. The method of claim 12, wherein the composition is able to decrease pro-inflammatory cytokines, increase anti-inflammatory cytokines, and/or reduce leukocyte infiltration.

17. The method of claim 12, wherein the composition is able to decrease a population of IL-17-producing T cells, increase a population of IL-10-producing T cells, and increase a population of regulatory T cells.

18. The method of claim 12, wherein the composition further comprises at least one pharmaceutically acceptable carrier, adjuvant, excipient, and/or diluent.

19. The method of claim 12, wherein the composition is administered via a route selected from the group consisting of oral, topical, transdermal, parenteral, subcutaneous, intranasal, intratracheal, intrabronchial, mucosal, intramuscular, intraperitoneal, intravitreal, and intravenous routes.

20. The method of claim 12, wherein the composition is in the form of a tablet, dragee, liquid, drop, suppository, capsule, caplet, or gelcap.


SEQUENCE LISTING (1 sequence)

SEQ ID NO: 1 - protein, 162 aa, Homo sapiens
MQEAGGRPGADCEVCKEFLNRFYKSLIDRGVNFSLDTIEKELISFCLDTKGKENRLCYYLGATKDAATKILSEVTRPMSVHMPAMKICEKLKKLDSQICELKYEKTLDLASVDLRKMRVAELKQILHSWGEECRACAEKTDYVNLIQELAPKYAATHPKTEL